(19) United States
(12) Patent Application Publication
(10) Pub. No.: US 2026/0112461 A1
(43) Pub. Date: Apr. 23, 2026

(54) SYSTEM AND METHOD FOR PREDICTING ODOR USING MACHINE LEARNING

(71) Applicants: Hyundai Motor Company, Seoul (KR); Kia Corporation, Seoul (KR); AJOU UNIVERSITY INDUSTRY-ACADEMIC COOPERATION FOUNDATION, Suwon (KR)

(72) Inventors: Shin Jong Lee, Hwaseong (KR); Young Jin Lee, Hwaseong (KR); Ha Young Yoon, Hwaseong (KR); Yoon Shin Kwak, Suwon (KR); Hyun Jung Shin, Suwon (KR)

(21) Appl. No.: 19/169,458

(22) Filed: Apr. 3, 2025

(30) Foreign Application Priority Data
Oct. 17, 2024 (KR) 10-2024-0142480

(51) Int. Cl. G16C 20/30; G06N 20/00; G16C 20/70

(52) U.S. Cl. CPC G16C 20/30; G06N 20/00; G16C 20/70

(57) ABSTRACT

A computer device comprises one or more processors, and one or more computer-readable media storing instructions that cause the computing device to perform operations, when executed by the one or more processors. The operations comprise generating basic data by deriving a main compound associated with each odor type from among compounds included in each of a plurality of gas samples, generating learning data by expanding a type of the main compound associated with each odor type using a similarity between the plurality of gas samples and a similarity between the compounds, learning an odor prediction model using the learning data, and predicting quality of an odor of an analysis target using the learned odor prediction model.

### CROSS-REFERENCE TO RELATED APPLICATION(S)

This application claims, under 35 U.S.C. § 119(a), the benefit of Korean Patent Application No. 10-2024-0142480 filed in the Korean Intellectual Property Office on Oct. 17, 2024, the disclosure of which is incorporated herein by reference in its entirety.

### BACKGROUND

### Field

The present disclosure relates to systems and methods for predicting an odor using machine learning.

### Background

Electronic noses, configured to recognize odors using patterns created by various combinations of sensors, have been used as a representative odor recognition technology. However, electronic noses have limitations. The development of sensors capable of accommodating various odors is insufficient, and an odor may be recognized as a different odor when a pattern changes due to a reduced lifespan of a sensor.

Additionally, the number of compounds for each type of odor, included in sample data for generating training data, may be unbalanced. Thus, there is a limitation in that a prediction model may have degraded performance when learning the prediction model using the number of compounds for each type of odor.

### SUMMARY

An aspect of the present disclosure provides systems and methods using gas chromatography-mass spectrometry (GC-MS) data, the system and method capable of resolving an issue associated with misrecognition due to a reduced sensor lifespan of an electronic nose according to the related art, and objectively and accurately diagnosing a type of an odor.

According to an exemplary embodiment, systems and methods are provided capable of an issue associated with imbalance of training data and increasing performance of a prediction model by associating compounds having similar chemical structures with types of odors to expand learning data.

In one aspect, a computer device is provided comprising: 1) one or more processors; and 2) one or more computer-readable media configured to store instructions that, when executed by the processor, are configured to cause the computing device to: i) generate basic data by deriving a main compound associated with each odor type from among compounds included in each of a plurality of gas samples; ii) generate learning data by expanding a type of the main compound associated with each odor type using a similarity between the plurality of gas samples and a similarity between the compounds; iii) learn an odor prediction model using the learning data; and iv) predict quality of an odor of an analysis target using the learned odor prediction model.

According to an exemplary embodiment, there is provided a computer device including one or more processors, and one or more computer-readable media storing instructions that cause the computing device to perform operations, when executed by the one or more processors. The operations may include generating basic data by deriving a main compound associated with each odor type from among compounds included in each of a plurality of gas samples, generating learning data by expanding a type of the main compound associated with each odor type using a similarity between the plurality of gas samples and a similarity between the compounds, learning an odor prediction model using the learning data, and predicting quality of an odor of an analysis target using the learned odor prediction model.

According to an exemplary embodiment, there is provided a method of implementing a computer, the method including generating basic data by deriving a main compound associated with each odor type from among compounds included in each of a plurality of gas samples, generating learning data by expanding a type of the main compound associated with each odor type using a similarity between the plurality of gas samples and a similarity between the compounds, learning an odor prediction model using the learning data, and diagnosing an odor of an analysis target using the learned odor prediction model.

According to an exemplary embodiment, GC-MS data of an analysis target may be preprocessed to extract a chemical component included in an odor of the analysis target, and a type of the odor may be diagnosed based on the extracted chemical component, thereby resolving an issue associated with misrecognition due to a reduced sensor lifespan of an electronic nose according to the related art.

According to an exemplary embodiment, a type of a main compound associated with an odor may be expanded using a similarity between gas samples and a similarity between compounds, thereby resolving an issue associated with imbalance between pieces of data to increase performance of a prediction model.

As referred to herein, in aspects, a main compound associated with an odor type is a single compound (discrete compound) that will be present in the greater amount (e.g. greater percent by volume or weight) relative to any other single compound for an odor type. In aspects, a main compound will constitute a substantial portion of an odor, e.g. up to or at least 10, 20, 30, 40, 50, 60, 70, 80 or 90 percent (volume or weight) of the total compounds associated with an odor type will be the main compound.

### BRIEF DESCRIPTION OF DRAWINGS

The above and other aspects, features, and advantages of the present disclosure will be more clearly understood from the following detailed description, taken in conjunction with the accompanying drawings, in which:

FIG. 1 is a flowchart illustrating a method of diagnosing an odor, according to an exemplary embodiment of the present disclosure;

FIG. 2 is a flowchart illustrating operation S120 of FIG. 1 in more detail, according to an exemplary embodiment of the present disclosure;

FIG. 3 is a flowchart illustrating operation S121 of FIG. 2 in more detail, according to an exemplary embodiment of the present disclosure;

FIG. 4 is a flowchart illustrating operation S122 of FIG. 2 in more detail, according to an exemplary embodiment of the present disclosure;

FIG. 5 is a flowchart illustrating operation S110 of FIG. 1 in more detail, according to an exemplary embodiment of the present disclosure;

FIG. 6 is an exemplary diagram illustrating a method of generating learning data, according to an exemplary embodiment of the present disclosure;

FIGS. 7-9 are exemplary diagrams illustrating a method of calculating a similarity between a plurality of gas samples, according to exemplary embodiments of the present disclosure;

FIG. 10 is an exemplary diagram illustrating a method of calculating a similarity between compounds, according to an exemplary embodiment of the present disclosure;

FIG. 11 is an exemplary diagram illustrating a method of generating learning data by expanding a type of a main compound, according to an exemplary embodiment of the present disclosure;

FIGS. 12A-12B are diagrams illustrating an effect of an odor prediction model learned, according to exemplary embodiments of the present disclosure; and

FIG. 13 illustrates a block diagram illustrating a computing system, according to an exemplary embodiment of the present disclosure.

### DETAILED DESCRIPTION

The following Detailed Description is merely provided by way of example and not of limitation. Furthermore, there is no intention to be bound by any expressed or implied theory presented in the preceding background or in the following Detailed Description.

Reference will now be made in detail to various exemplary embodiments of the subject matter, examples of which are illustrated in the accompanying drawings. While various embodiments are discussed herein, it will be understood that they are not intended to limit to these embodiments. On the contrary, the presented embodiments are intended to cover alternatives, modifications, and equivalents, which may be included within the spirit and scope of the various embodiments as defined by the appended claims. Furthermore, in this Detailed Description, numerous specific details are set forth in order to provide a thorough understanding of embodiments of the present subject matter. However, embodiments may be practiced without these specific details. In other instances, well known methods, procedures, components, and circuits have not been described in detail as not to unnecessarily obscure aspects of the described embodiments.

Some portions of the detailed descriptions which follow are presented in terms of procedures, logic blocks, processing, and other symbolic representations of operations on data within an electrical device. These descriptions and representations are the means used by those skilled in the data processing arts to most effectively convey the substance of their work to others skilled in the art. In the present application, a procedure, logic block, process, or the like, is conceived to be one or more self-consistent procedures or instructions leading to a desired result. The procedures are those requiring physical manipulations of physical quantities. Usually, although not necessarily, these quantities may take the form of electrical or magnetic signals capable of being stored, transferred, combined, compared, and otherwise manipulated in an electronic system, device, and/or component.

It should be borne in mind, however, that these and similar terms are to be associated with the appropriate physical quantities and are merely convenient labels applied to these quantities. Unless specifically stated otherwise as apparent from the following discussions, it is appreciated that throughout the description of embodiments, discussions utilizing terms such as “determining,” “communicating,” “taking,” “comparing,” “monitoring,” “calibrating,” “estimating,” “initiating,” “providing,” “receiving,” “controlling,” “transmitting,” “isolating,” “generating,” “aligning,” “synchronizing,” “identifying,” “maintaining,” “displaying,” “switching,” or the like, refer to the actions and processes of an electronic item such as: a processor, a sensor processing unit (SPU), a processor of a sensor processing unit, an application processor of an electronic device/system, or the like, or a combination thereof. The item manipulates and transforms data represented as physical (electronic and/or magnetic) quantities within the registers and memories into other data similarly represented as physical quantities within memories or registers or other such information storage, transmission, processing, or display components.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. These terms are merely intended to distinguish one component from another component, and the terms do not limit the nature, sequence or order of the constituent components. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Throughout the specification, unless explicitly described to the contrary, the word “comprise” and variations such as “comprises” or “comprising” will be understood to imply the inclusion of stated elements but not the exclusion of any other elements. In addition, the terms “unit”, “-er”, “-or”, and “module” described in the specification mean units for processing at least one function and operation, and can be implemented by hardware components or software components and combinations thereof.

Although exemplary embodiment is described as using a plurality of units to perform the exemplary process, it is understood that the exemplary processes may also be performed by one or plurality of modules. Additionally, it is understood that the term controller/control unit refers to a hardware device that includes a memory and a processor and is specifically programmed to execute the processes described herein. The memory is configured to store the modules and the processor is specifically configured to execute said modules to perform one or more processes which are described further below.

Further, the control logic of the present disclosure may be embodied as non-transitory computer readable media on a computer readable medium containing executable program instructions executed by a processor, controller or the like. Examples of computer readable media include, but are not limited to, ROM, RAM, compact disc (CD)-ROMs, magnetic tapes, floppy disks, flash drives, smart cards and optical data storage devices. The computer readable medium can also be distributed in network coupled computer systems so that the computer readable media is stored and executed in a distributed fashion, e.g., by a telematics server or a Controller Area Network (CAN).

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about”.

Embodiments described herein may be discussed in the general context of processor-executable instructions residing on some form of non-transitory processor-readable medium, such as program modules, executed by one or more computers or other devices. Generally, program modules include routines, programs, objects, components, data structures, etc., that perform particular tasks or implement particular abstract data types. The functionality of the program modules may be combined or distributed as desired in various embodiments.

In the figures, a single block may be described as performing a function or functions; however, in actual practice, the function or functions performed by that block may be performed in a single component or across multiple components, and/or may be performed using hardware, using software, or using a combination of hardware and software. To clearly illustrate this interchangeability of hardware and software, various illustrative components, blocks, modules, logic, circuits, and steps have been described generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example device vibration sensing system and/or electronic device described herein may include components other than those shown, including well-known components.

Various techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium comprising instructions that, when executed, perform one or more of the methods described herein. The non-transitory processor-readable data storage medium may form part of a computer program product, which may include packaging materials.

The non-transitory processor-readable storage medium may comprise random access memory (RAM) such as synchronous dynamic random access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, and/or executed by a computer or other processor.

Various embodiments described herein may be executed by one or more processors, such as one or more motion processing units (MPUs), sensor processing units (SPUs), host processor(s) or core(s) thereof, digital signal processors (DSPs), general purpose microprocessors, application specific integrated circuits (ASICs), application specific instruction set processors (ASIPs), field programmable gate arrays (FPGAs), a programmable logic controller (PLC), a complex programmable logic device (CPLD), a discrete gate or transistor logic, discrete hardware components, or any combination thereof designed to perform the functions described herein, or other equivalent integrated or discrete logic circuitry. The term “processor,” as used herein may refer to any of the foregoing structures or any other structure suitable for implementation of the techniques described herein. As employed in the subject specification, the term “processor” can refer to substantially any computing processing unit or device comprising, but not limited to comprising, single-core processors; single-processors with software multithread execution capability; multi-core processors; multi-core processors with software multithread execution capability; multi-core processors with hardware multithread technology; parallel platforms; and parallel platforms with distributed shared memory. Moreover, processors can exploit nano-scale architectures such as, but not limited to, molecular and quantum-dot based transistors, switches and gates, in order to optimize space usage or enhance performance of user equipment. A processor may also be implemented as a combination of computing processing units.

In addition, in some aspects, the functionality described herein may be provided within dedicated software modules or hardware modules configured as described herein. Also, the techniques could be fully implemented in one or more circuits or logic elements. A general purpose processor may be a microprocessor, but in the alternative, the processor may be any processor, controller, microcontroller, or state machine. A processor may also be implemented as a combination of computing devices, e.g., a combination of an SPU/MPU and a microprocessor, a plurality of microprocessors, one or more microprocessors in conjunction with an SPU core, MPU core, or any other such configuration. One or more components of an SPU or electronic device described herein may be embodied in the form of one or more of a “chip,” a “package,” an Integrated Circuit (IC).

According to exemplary embodiments, systems and methods for predicting an odor using machine learning are provided.

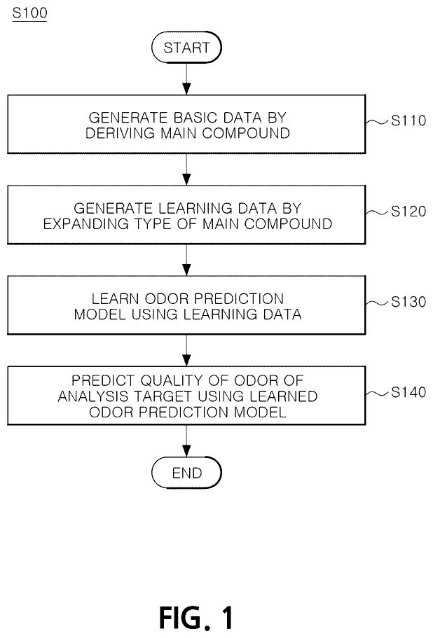

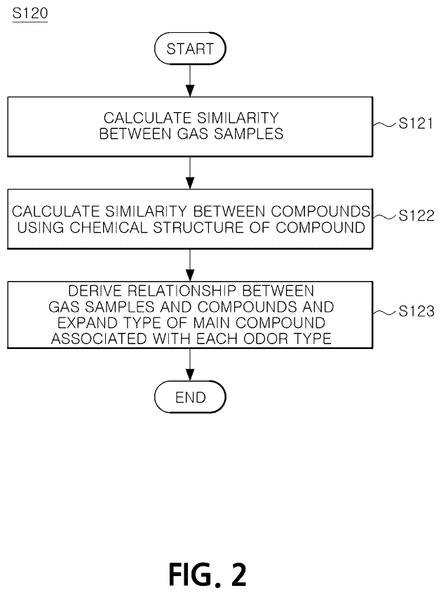

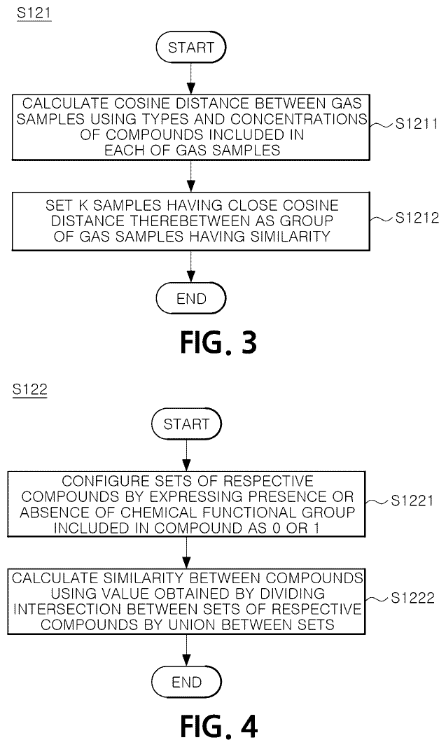

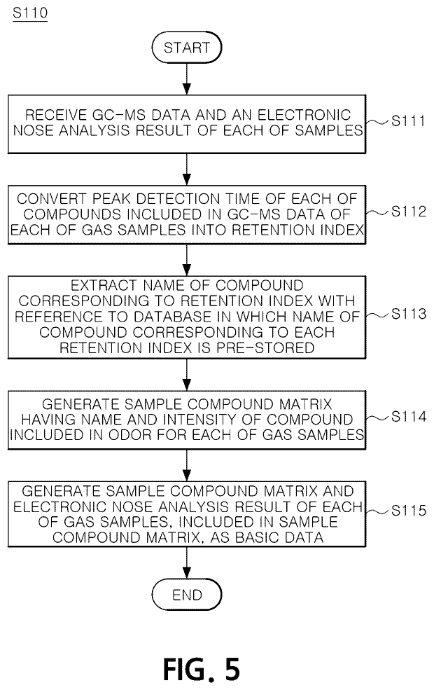

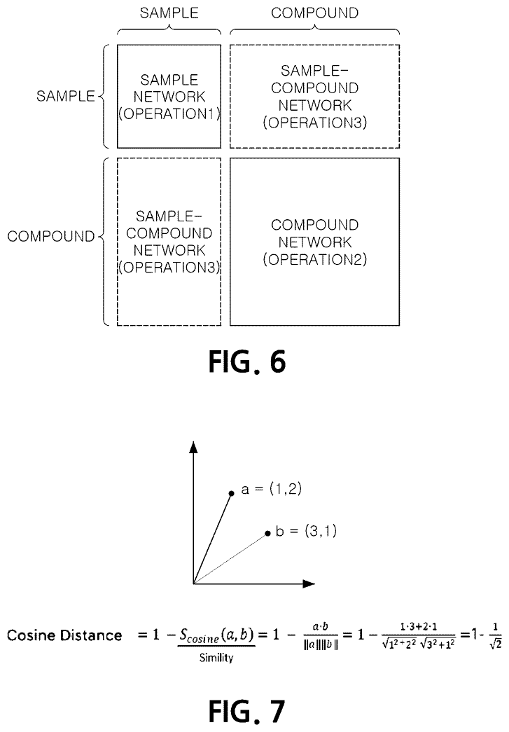

Referring now to FIGS. 1-5, a flowchart illustrating a method (S100) for diagnosing an odor is illustratively depicted, in accordance with an exemplary embodiment of the present disclosure.

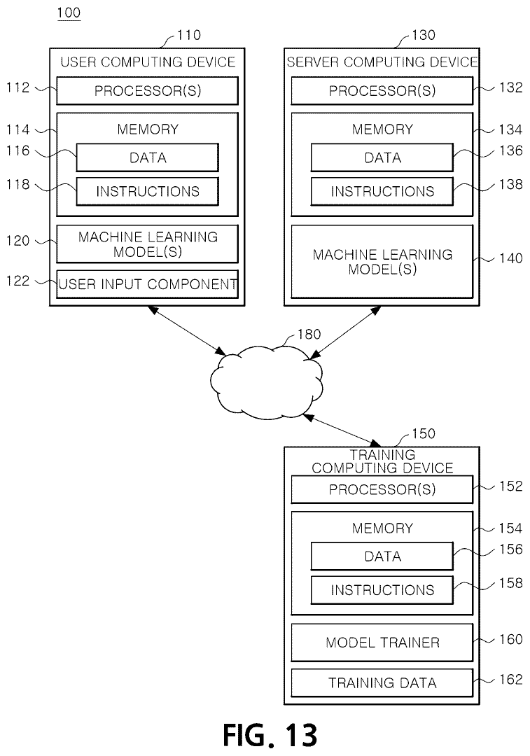

FIGS. 1-5 illustrate operations performed in a specific order for purposes of illustration and discussion. It is noted that the method of the present disclosure is not limited to the illustrated order or arrangement. Various operations of the method (S100) may be omitted, rearranged, combined, and/or adjusted in various manners without departing from the scope of the present disclosure. In addition, the method (S100) may be implemented by one or more computing devices, such as one or more computing devices illustrated in FIG. 13.

At S110, the method (S100) may comprise an operation (S110) of generating basic data by deriving a main compound associated with each odor type from among compounds included in each of a plurality of gas samples. According to an exemplary embodiment, the basic data may be configured by analyzing the significance and importance between compound components, detected from an electronic nose analysis result applied with an electronic sensor and gas chromatography-mass spectrometry (GC-MS) data for odors of the plurality of gas samples, to derive a main compound.

According to an exemplary embodiment, a main compound for each odor type may be selected as a multi-selected compound. The main compound for each odor type may be selected as a multi-selected compound using a statistical testing method, a machine learning algorithm, and/or via other suitable means. For example, a type of odor may comprise types of odors such as, e.g., a sour odor, a fruity/sweet odor, a burning plastic odor, an earthy/dusty odor, an oil/grease odor, a chemical agent odor, a leather odor, a grass odor, a rubber odor, a plastic odor, a tar/asphalt odor, a musty odor, a phenol/disinfectant odor, a fish/fishy odor, a sulfur/gas odor, and/or other suitable odor types. According to an exemplary embodiment, a plurality of main compounds may be selected for each odor type.

According to an exemplary embodiment, the systems and methods of the present disclosure may be configured to generate basic data by receiving GC-MS data and an electronic nose analysis result of each of a plurality of samples, and derive a main compound associated with each odor type (S111 to S115).

At S111, GC-MS data and an electronic nose analysis result of each of the plurality samples may be received.

At S112, a peak detection time of a compound included in the GC-MS data may be converted for each of a plurality of samples into a retention index (RI). The GC-MS data may comprise one or more peak detection times and intensities of various compounds included in an odor of an analysis target. According to an exemplary embodiment, the retention index may be obtained based on the GC-MS data.

According to an exemplary embodiment, the above-described retention index may be obtained using a method of converting the data into independent constants of components and using the independent constants for qualitative analysis. The method may be performed by, for example, calculating a relative position of a peak detection time of a target component, based on a peak detection time of an alkane compound. Thereafter, a name of a compound may be extracted, the compound corresponding to the converted retention index with reference to a database in which the name of compound corresponding to the each retention index is pre-stored (S113), and a sample compound matrix may be generated having a name and an intensity of a compound included in an odor for each of the plurality of samples (S114).

The sample compound matrix may be in the form of a table comprising names and intensities of compounds (e.g., Compound 1 to Compound N) included in an odor for each of a plurality of samples (e.g., Sample 1 to Sample M).

According to an exemplary embodiment, the sample compound matrix and an electronic nose analysis result of each of the plurality of samples, included in the sample compound matrix, may be generated as the basic data (S115).

According to an exemplary embodiment, the electronic nose may comprise a device configured to recognize a type of odor using a sensor pattern created by various combinations of sensors. According to an analysis result of the electronic nose, a type of odor may be diagnosed using the sensor pattern. The one or more sensors may be implemented by combining a plurality of sensors for each detection gas and concentration. According to an exemplary embodiment, the one or more sensors may comprise, for example, three PID sensors (for detecting VOCs), two E.C. sensors (for detecting H₂S), ten S.C. sensors (for detecting NH₃, alcohol, and CH₄), one IR sensor (for detecting CO₂), and one T&H sensor (for detecting hydrocarbons). It should be noted that specific examples of the above-described sensors are only for assisting in understanding of the present disclosure, and are not limited to the above-described sensors.

According to an exemplary embodiment, the sample compound matrix may comprise at least two or more compounds for each of the plurality of samples.

According to an exemplary embodiment, a main compound may be selected from among compounds included in each of the plurality of gas samples, based on an electronic nose analysis result of each of the plurality of gas samples, and the electronic nose analysis result and the main compound may be generated as the basic data.

According to an exemplary embodiment, a compound may be selected. According to an exemplary embodiment, the compound may be selected repeatedly in at least two feature selection algorithms, as the main compound.

Using all raw data as features during machine learning may be highly inefficient in terms of computational power and memory. Thus, significant data may be preferably selected and used using a feature selection algorithm. The feature selection algorithm may comprise a chi-square filter, correlation-based feature selection, decision tree-based feature selection, and forward selection. It is noted that other features may be incorporated while maintaining the spirit and functionality of the present disclosure.

According to an exemplary embodiment, when there are a small number of gas samples having a specific odor, machine learning may not be sufficiently performed due to a small number of main compounds selected related to the odor. For example, due to a small number of gas samples having a sour odor, a fruity/sweet odor, and a burning plastic odor, it may be difficult to configure the gas samples as leaning data for machine learning. When an amount of data is insufficient, prediction performance may be degraded. Thus, it may be necessary to increase prediction accuracy of an odor prediction model by expanding the basic data. Accordingly, in the present disclosure, a method of configuring leaning data by expanding a type of a main compound of basic data in order to increase prediction performance of an odor prediction model is presented.

Referring back to FIG. 1, at S120, the method (S100) may comprise an operation of generating learning data by expanding a type of the main compound associated with each odor type using a similarity between the plurality of gas samples and a similarity between the compounds. More specifically, an operation (S120) of generating learning data may further comprise an operation (S121) of calculating the similarity between the plurality of gas samples by comparing types and concentrations of the compounds, included in each of the plurality of gas samples, to each other, an operation (S122) of calculating the similarity between the compounds using a chemical structure of the compound, and an operation (S123) of deriving a relationship between the plurality of gas samples and the compounds using the similarity between the plurality of gas samples and the similarity between the compounds, and expanding the type of the main compound associated with each odor type using the relationship between the plurality of gas samples and the compounds.

According to an exemplary embodiment, the operation (S121) of calculating the similarity between the plurality of gas samples may further comprise an operation (S1211) of calculating a cosine distance between the plurality of gas samples using the types and concentrations of the compounds included in each of the plurality of gas samples, and an operation (S1212) of setting k samples having a close cosine distance therebetween as a group of gas samples having a similarity.

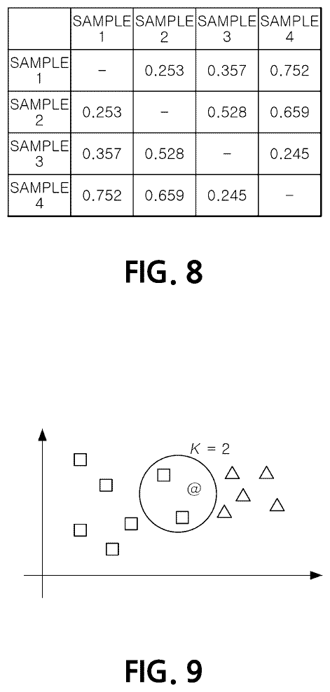

FIG. 6 is an exemplary diagram illustrating a method of generating learning data, in accordance with an exemplary embodiment of the present disclosure.

Referring to FIG. 6, in a first operation, a sample network defining a correlation between a plurality of gas samples may be formed. In a second operation, a compound network defining a correlation of compounds included in the plurality of gas samples may be formed. In a third operation, learning data may be generated by defining a correlation between the gas sample network and the compound network. The generated learning data may comprise a larger number of main compounds by expanding types of main compounds associated with each odor type included in basic data. According to an exemplary embodiment, the number of pieces of data may be expanded using limited gas samples, such that a prediction model may have improved performance using limited data.

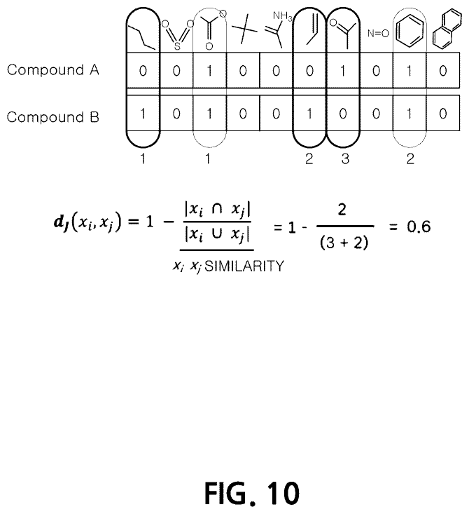

FIGS. 7-9 are exemplary diagrams illustrating a method of calculating a similarity between a plurality of gas samples, in accordance with exemplary embodiments of the present disclosure.

According to an exemplary embodiment, a sample network may be configured. The sample network may indicate a correlation between gas samples using data on a type and a concentration of a compound included in GC-MS data. A similarity between the gas samples may be calculated. More specifically, a similarity between the gas samples may be derived by measuring a cosine distance between the gas samples in consideration of types of the gas samples and components detected in the gas samples.

Referring to FIG. 7, a cosine distance between Gas Sample a and Gas Sample b may be derived by the equation indicated in FIG. 7 and reproduced below as Equation 1.

$$Cosine Distance = 1 - \frac{{S}_{c o s i n e} ( a , b )}{Simility} = 1 - \frac{a \cdot b}{‖ a ‖ ‖ b ‖} \qquad Equation 1$$

According to an exemplary embodiment, the cosine distance may be obtained using a method of measuring an angle difference between two vectors, and may be a value indicating an angle similarity between the two vectors based on a cosine similarity. According to an exemplary embodiment, coordinates of a sample may comprise data included in the GC-MS data, and the coordinates of the sample may be defined as a type of a detected component and a detected concentration.

For example, when the coordinates are defined as Sample a=(1,2), Sample b=(3,1), a cosine distance between Sample a and Sample b may be calculated by Equation 2 below.

$$1 - \frac{a * b}{‖ a ‖ * ‖ b ‖} = 1 - \frac{1 * 3 + 2 * 1}{\sqrt{{1}^{2} + {2}^{2}} * \sqrt{{3}^{2} + {1}^{2}}} = 1 - \frac{1}{\sqrt{2}} \qquad Equation 2$$

A sample included in the GC-MS data may be expressed as a table including 200 pieces of data on a type of a detected component and a detected concentration. A similarity between samples may indicate whether detected components overlap each other and whether concentrations of overlapping components are similar to each other. However, a large number of pieces of data may be included in one gas sample. Thus, when a generally used squared Euclidean distance is applied, it may be difficult to measure a distance.

Accordingly, in an exemplary embodiment of the present disclosure, a similarity may be measured based on an angle difference rather than a vector length between pieces of data by applying a cosine distance, thereby effectively measuring a distance between the pieces of data in a high-dimensional space.

Referring to FIG. 8, a table including numerical values, obtained by respectively calculating similarities between a plurality of gas samples, in accordance with an exemplary embodiment of the present disclosure.

In accordance with FIG. 9, a system and method of the present disclosure may set k samples having a close cosine distance therebetween as a group of gas samples having a similarity.

According to an exemplary embodiment, a k-nearest neighbor (k-NN) method may be a method of making classifications such that k pieces of close training data are configured, and finding a correlation, and may be a method of connecting between samples (that is, nodes having strong connectivity) having a close distance therebetween. The algorithm may be simple and easy to implement, such that it may be effective when dealing with semi-supervised learning type data, a mixture of labeled data and unlabeled data.

However, an estimated result may vary according to the number (k) of pieces of close data for finding the correlation, and thus it may be significant to set an appropriate k value. In FIG. 9, according to an exemplary embodiment, k may be set to 2 to define a relationship, but may be set within various ranges in some cases.

According to an exemplary embodiment, the operation (S122) of calculating the similarity between the compounds using the chemical structure of the compound may comprise an operation (S1221) of configuring sets of respective compounds by expressing presence or absence of a chemical functional group included in a compound as 0 or 1; and an operation (S1222) of calculating the similarity between the compounds using a value obtained by dividing an intersection between the sets of the respective compounds by a union between the sets.

A method of calculating a similarity between compounds may comprise a method of supplementing the number of pieces of label imbalance odor data using a characteristic that “compounds having a similar structure will have a similar property or odor,” and a connection between the compounds may be defined using a similarity between chemical structures of the compounds. This may be because it is necessary to quantitatively measure a similarity between two molecules when measuring a distance between pieces of compound data.

According to an exemplary embodiment, compounds may be converted using a molecular fingerprint method, and a similarity between two molecules may be quantitatively measures using a Jaccard distance.

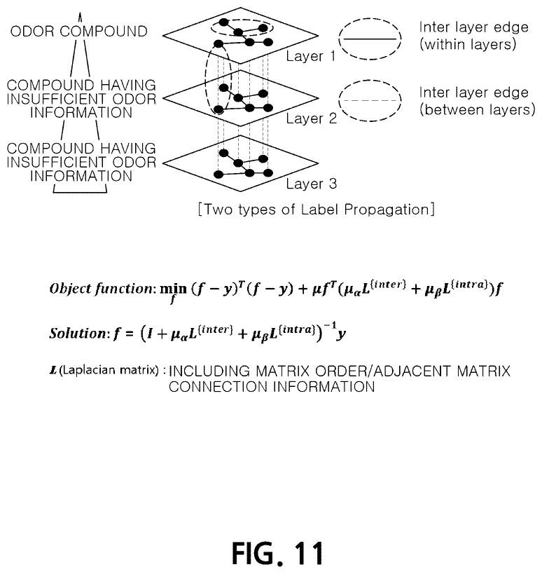

Referring to FIG. 10, a presence or absence of each of functional groups included in Compound A and Compound B may be indicated by 0 or 1, and sets of respective compounds may be configured. An intersection between a set of Compound A and a set of Compound B may be calculated as 2, and a union between the set of Compound A and the set of Compound B may be calculated as 5. In addition, a similarity between Compound A and Compound B may be calculated as 0.6.

According to an exemplary embodiment, a relationship between a plurality of gas samples and compounds may be derived using a similarity between the plurality of gas samples and a similarity between the compounds, and a type of a main compound associated with each odor type may be expanded using the relationship between the plurality of gas samples and the compounds.

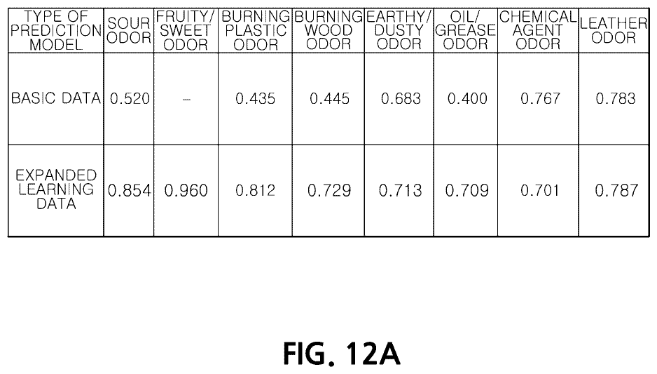

Referring now to FIG. 11, the systems and methods of the present disclosure may be configured to resolve an issue associated with imbalance of label data by expanding the number of compounds associated with an odor using a label propagation method.

The label propagation method may comprise a method of expanding label data by transferring label information that is clearly known to a compound having high similarity one step at a time. For example, when a number of gas samples is 26 and a number of compounds is 373, a total number of nodes may be 399. Additionally, a value of a network density calculated to verify whether pieces of data have a close connection may be 0.06, from which it may be confirmed that respective networks are sparsely connected to each other.

According to an exemplary embodiment, the systems and methods of the present disclosure may be configured to obtain a solution using a minimization function in a definition formula of semi-supervised learning to minimize information loss due to a large number of pieces of unlabeled data, and may be configured to configure an equation by reflecting a label propagation direction (inter and intra) factor to increase the number of compounds associated with an odor. According to an exemplary embodiment, an objective function may be configured by Equation 3, and a solution function may be configured by Equation 4.

$${min ( f - y )}^{T} ( f - y ) + μ {f}^{T} ( {μ}_{α} {L}^{\{ inter \}} + {μ}_{β} {L}^{\{ inter \}} ) f \qquad Equation 3$$

$$f = {( I + {μ}_{α} {L}^{\{ inter \}} + {μ}_{β} {L}^{\{ inter \}} )}^{- 1} ) y \qquad Equation 4$$

In Equations 3 and 4, L may include matrix order/adjacent matrix connection information, an inter direction may refer to a connection in the same layer, and an intra direction may refer to a connection between adjacent measurements.

The systems and methods of the present disclosure may be configured to expand a type of labeled compound associated with an odor in the same layer using the objective function of Equation 3 and the solution function of Equation 4, may be configured to define a connection relationship with a compound having insufficient odor information, and may be configured to expand the number of main compounds related to the odor, thereby configuring learning data.

Referring back to FIG. 1, at S130, the method (S100) may comprise an operation of learning an odor prediction model using the learning data.

According to an exemplary embodiment, in S140, the method (S100) may comprise an operation of predicting quality of an odor of an analysis target using the learned odor prediction model.

A type of the odor included in the analysis target may be identified and diagnosed using the system and method of the present disclosure, thereby predicting the quality of the odor.

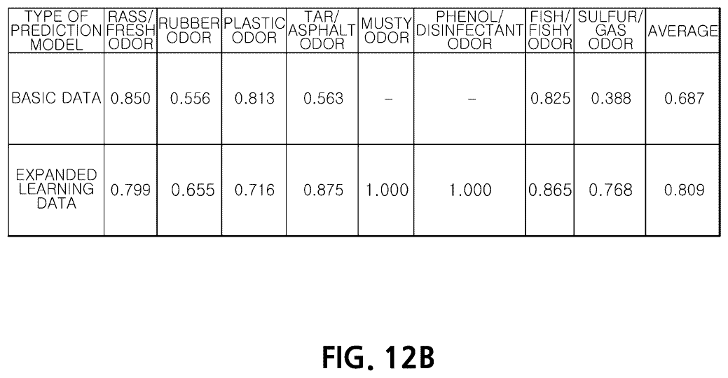

FIGS. 12A-12B are diagrams illustrating an effect of an odor prediction model learned, in accordance with exemplary embodiments of the present disclosure.

Referring to FIG. 12A and FIG. 12B, it may be confirmed that a model has improved prediction performance in a case in which the odor prediction model is learned using learning data configured by expanding a type of a main compound, as compared to a case in which the odor prediction model is learned using basic data including only a main compound associated with an odor. According to an exemplary embodiment, in order to diagnose prediction performance of the model, the performance may be quantitatively identified using an area under the ROC curve (AUC), and the model may have excellent performance as the AUC is closer to 1.

Referring now to FIG. 13, a block diagram of an exemplary computing system 100 configured to easily diagnose and predict quality of an odor is illustratively depicted, in accordance with an exemplary embodiment of the present disclosure.

The system 100 may be provided as only one example. Other computing systems including different components may be additionally or alternatively used for the system 100. The system 100 may comprise a user computing device 110 communicably connected via a network 180, a server computing system 130, and a training computing system 150.

The user computing device 110 may comprise any type of computing device, such as a personal computing device (for example, a laptop or a desktop), a mobile computing device (for example, a smartphone or tablet), a game console or controller, a wearable computing device, an embedded computing device, and/or any other type of computing device.

The user computing device 110 may comprise one or more processors 112 and a memory 114. The one or more processors 112 may be any suitable processing devices (for example, a processor core, a microprocessor, an ASIC, an FPGA, a controller, a microcontroller, or the like), and may be one processor or a plurality of operably connected processors. The memory 114 may comprise one or more non-transitory computer-readable storage media such as RAM, ROM, EEPROM, EPROM, a flash memory device, a magnetic disk, and combinations thereof. The memory 114 may be configured to store data 116 and instructions 118 that are executed by processor 112 and cause the user computing device 110 to perform operations.

In some implementations, the user computing device 110 may be configured to store or include one or more machine learning models 120, such as the odor prediction model set forth herein. For example, the machine learning model 120 may be or comprise various machine learning models, such as a neural network (for example, a deep neural network) or other types of machine learning models including a nonlinear model and/or a linear model. The neural network may comprise a feedforward neural network, a recurrent neural network (for example, a long-short term memory recurrent neural network), a convolutional neural network, and/or other types of neural networks.

In some implementations, the one or more machine learning models 120 may be received from the server computing system 130 via the network 180, stored in the user computing device memory 114 and then used or implemented by the one or more processors 112. In some implementations, the user computing device 110 may implement multiple parallel instances of a single machine learning model 120.

Additionally or alternatively, the one or more machine learning models 140 may be included in the server computing system 130 communicating with the user computing device 110 according to a client-server relationship, or otherwise stored and implemented in the server computing system 130. For example, the machine learning model 140 may be implemented by the server computing system 140 as a portion of a web service. Thus, the one or more models 120 may be stored and implemented in the user computing device 110, and/or the one or more models 140 may be stored and implemented in server computing system 130.

The user computing device 110 may comprise one or more user input components 122 receiving a user input. For example, the user input component 122 may be a touch-sensitive component (for example, a touch-sensitive display screen or touch pad) that is sensitive to the touch of a user input object (for example a finger or a stylus). The touch-sensitive component may serve to implement a virtual keyboard. Other exemplary user input components may include a microphone, a traditional keyboard, a camera, or other means by which a user may provide a user input.

The server computing system 130 may comprise one or more processors 132 and a memory 134. The one or more processors 132 may comprise any suitable processing devices (for example, a processor core, a microprocessor, an ASIC, an FPGA, a controller, a microcontroller, or the like), and may comprise one processor or a plurality of operably connected processors. The memory 134 may comprise one or more non-transitory computer-readable storage media such as RAM, ROM, EEPROM, EPROM, a flash memory device, a magnetic disk, and combinations thereof. The memory 134 may be configured to store data 136 and instructions 138 that are executed by the processor 132 and cause the server computing system 130 to perform operations.

In some implementations, the server computing system 130 may comprise one or more server computing devices or may be implemented by the one or more server computing devices. When the server computing system 130 comprise a plurality of server computing devices, the server computing devices may be configured to operate according to a sequential computing architecture, a parallel computing architecture, or some combination thereof.

As described above, the server computing system 130 may be configured to store or include one or more machine learning models 140. For example, the model 140 may be or include various machine learning models such as odor prediction models. Examples of the machine learning model may be a neural network or other multilayer nonlinear models. Examples of the neural network include a feedforward neural network, a deep neural network, a recurrent neural network, and a convolutional neural network.

The user computing device 110 and/or the server computing system 130 may be configured to train the model 120 and/or 140 through interaction with the training computing system 150 communicatively connected via the network 180. The training computing system 150 may be separated from the server computing system 130 or may be a portion of the server computing system 130.

The training computing system 150 may comprise one or more processors 152 and a memory 154. The one or more processors 152 may comprise any suitable processing device (for example, a processor core, a microprocessor, an ASIC, an FPGA, a controller, a microcontroller, or the like), and may be one processor or a plurality of operably connected processors. The memory 154 may comprise one or more non-transitory computer-readable storage media, such as RAM, ROM, EEPROM, EPROM, a flash memory device, a magnetic disk, and combinations thereof. The memory 154 may be configured to store data 156 and instructions 158 that are executed by the processor 152 and cause the training computing system 150 to perform operations. In some implementations, the training computing system 150 may comprise one or more training computing devices or may be implemented by the one or more training computing devices.

The training computing system 150 may comprise a model trainer 160 configured to train the machine learning model 120 and/or 140 to be stored in the user computing device 110 and/or server computing system 130 using various training or learning techniques, such as backpropagation of an error, for example. According to some exemplary embodiments, performing backpropagation of an error may comprise performing truncated backpropagation over time. The model trainer 160 may be configured to perform multiple generalization techniques (for example, weight reduction, dropout, or the like) to improve generalization capability of the model to be trained.

In particular, the model trainer 160 may be configured to train the machine learning model 120 and/or 140, based on a set of training data 162. The training data 162 may comprise, for example, information on a compound labeled for each type of odor.

The model trainer 160 may comprise a computer logic used to provide a desired function. The model trainer 160 may be implemented in hardware controlling a general-purpose processor, firmware, and/or software. For example, in some implementations, the model trainer 160 may comprise a program file stored in a storage device, loaded into a memory, and executed by one or more processors. In other implementations, the model trainer 160 may comprise one or more sets of computer-executable instructions stored in a RAM hard disk or a type of computer-readable storage medium such as an optical or magnetic medium.

The network 180 may comprise any type of communication network, such as a short-range network (for example, intranet), a wide-area network (for example, Internet), or some combination thereof, and may include any number of wired or wireless links. In general, communication via the network 180 may be performed via any type of wired and/or wireless connection using various communication protocols (for example, TCP/IP, HTTP, SMTP, or FTP), encoding or a format (for example, HTML or XML) and/or a protection scheme (for example, VPN, secure HTTP, or SSL).

FIG. 13 illustrates an exemplary computing system that may be used to implement the present disclosure. Other computing systems may also be used. For example, in some implementations, a user computing device 110 may comprise a model trainer 160 and a training dataset 162. In such an implementation, models 120 may be trained and used locally in the user computing device 110. Any components illustrated as being included in one of a device 102, a system 130, and/or a system 150 may instead be included in one or both of the device 102, the system 130, and/or the system 150.

What has been described above includes examples of the subject disclosure. It is, of course, not possible to describe every conceivable combination of components or methodologies for purposes of describing the subject matter, but it is to be appreciated that many further combinations and permutations of the subject disclosure are possible. Accordingly, the claimed subject matter is intended to embrace all such alterations, modifications, and variations that fall within the spirit and scope of the appended claims.

In particular and in regard to the various functions performed by the above described components, devices, systems and the like, the terms (including a reference to a “means”) used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter.

The aforementioned systems and components have been described with respect to interaction between several components. It can be appreciated that such systems and components can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components. Any components described herein may also interact with one or more other components not specifically described herein.

In addition, while a particular feature of the subject innovation may have been disclosed with respect to only one of several implementations, such feature may be combined with one or more other features of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements. Thus, the embodiments and examples set forth herein were presented in order to best explain various selected embodiments of the present invention and its particular application and to thereby enable those skilled in the art to make and use embodiments of the invention. However, those skilled in the art will recognize that the foregoing description and examples have been presented for the purposes of illustration and example only. The description as set forth is not intended to be exhaustive or to limit the embodiments of the invention to the precise form disclosed.

What is claimed is:

1. A computer device comprising:
1) one or more processors; and
2) one or more computer-readable media configured to store instructions that, when executed by the processor, are configured to cause the computing device to:
i) generate basic data by deriving a main compound associated with each odor type from among compounds included in each of a plurality of gas samples;
ii) generate learning data by expanding a type of the main compound associated with each odor type using a similarity between the plurality of gas samples and a similarity between the compounds;
iii) learn an odor prediction model using the learning data; and
iv) predict quality of an odor of an analysis target using the learned odor prediction model.

2. The computer device of claim 1, wherein the instructions, when executed by the processor, are further configured to cause the computing device to calculate the similarity between the plurality of gas samples by comparing types and concentrations of the compounds, included in each of the plurality of gas samples, to each other.

3. The computing device of claim 2, wherein the instructions, when executed by the processor, are further configured to cause the computing device to:
calculate a cosine distance between the plurality of gas samples using the types and concentrations of the compounds included in each of the plurality of gas samples; and
set k samples having a close cosine distance therebetween as a group of gas samples having a similarity.

4. The computing device of claim 1, wherein the instructions, when executed by the processor, are further configured to cause the computing device to calculate the similarity between the compounds using a chemical structure of the compound.

5. The computing device of claim 4, wherein the instructions, when executed by the processor, are further configured to cause the computing device to:
configure sets of respective compounds by expressing presence or absence of a chemical functional group included in a compound as 0 or 1; and
calculate the similarity between the compounds using a value obtained by dividing an intersection between the sets of the respective compounds by a union between the sets.

6. The computing device of claim 1, wherein the instructions, when executed by the processor, are further configured to cause the computing device to:
derive a relationship between the plurality of gas samples and the compounds using the similarity between the plurality of gas samples and the similarity between the compounds; and
expand the type of the main compound associated with each odor type using the relationship between the plurality of gas samples and the compounds.

7. The computing device of claim 1, wherein:
the instructions, when executed by the processor, are further configured to cause the computing device to receive gas chromatography-mass spectrometry (GC-MS) data and an electronic nose analysis result of each of the plurality of gas samples, and
the electronic nose analysis result comprises a type of an odor of each of the plurality of gas samples.

8. The computing device of claim 7, wherein the instructions, when executed by the processor, are further configured to cause the computing device to:
convert a peak detection time of each of compounds included in the GC-MS data of each of the plurality of gas samples into a retention index;
extract a name of a compound corresponding to the retention index with reference to a database in which a name of a compound corresponding to each retention index is pre-stored;
generate a sample compound matrix (SCM) having a name and an intensity of a compound included in an odor for each of the plurality of gas samples; and
generate the sample compound matrix and the electronic nose analysis result of each of the plurality of gas samples, included in the sample compound matrix, as the basic data.

9. The computing device of claim 8, wherein:
the sample compound matrix comprises at least two compounds for each of the plurality of samples, and
the instructions, when executed by the processor, are further configured to cause the computing device to:
select a main compound from among the compounds included in each of the plurality of gas samples, based on the electronic nose analysis result of each of the plurality of gas samples; and
generate the electronic nose analysis result and the main compound as the basic data.

10. The computing device of claim 9, wherein the instructions, when executed by the processor, are further configured to cause the computing device to select a compound, selected repeatedly in at least two feature selection algorithms, as the main compound.

11. A method of implementing a computer, the method comprising:
generating basic data by deriving a main compound associated with each odor type from among compounds included in each of a plurality of gas samples;
generating learning data by expanding a type of the main compound associated with each odor type using a similarity between the plurality of gas samples and a similarity between the compounds;
learning an odor prediction model using the learning data; and
predicting quality of an odor of an analysis target using the learned odor prediction model.

12. The method of claim 11, wherein the generating the learning data comprises calculating the similarity between the plurality of gas samples by comparing types and concentrations of the compounds, included in each of the plurality of gas samples, to each other.

13. The method of claim 12, wherein the calculating the similarity between the plurality of gas samples comprises:
calculating a cosine distance between the plurality of gas samples using the types and concentrations of the compounds included in each of the plurality of gas samples; and
setting k samples having a close cosine distance therebetween as a group of gas samples having a similarity.

14. The method of claim 11, wherein the generating the learning data comprises calculating the similarity between the compounds using a chemical structure of the compound.

15. The method of claim 14, wherein the calculating the similarity between the compounds comprises:
configuring sets of respective compounds by expressing presence or absence of a chemical functional group included in a compound as 0 or 1; and
calculating the similarity between the compounds using a value obtained by dividing an intersection between the sets of the respective compounds by a union between the sets.

16. The method of claim 11, wherein the generating the learning data comprises:
deriving a relationship between the plurality of gas samples and the compounds using the similarity between the plurality of gas samples and the similarity between the compounds; and
expanding the type of the main compound associated with each odor type using the relationship between the plurality of gas samples and the compounds.

17. The method of claim 11, wherein:
the generating the basic data comprises receiving GC-MS data and an electronic nose analysis result of each of the plurality of gas samples, and
the electronic nose analysis result comprises a type of an odor of each of the plurality of gas samples.

18. The method of claim 17, wherein the generating the basic data comprises:
converting a peak detection time of each of compounds included in the GC-MS data of each of the plurality of gas samples into a retention index;
extracting a name of a compound corresponding to the retention index with reference to a database in which a name of a compound corresponding to each retention index is pre-stored;
generating a sample compound matrix (SCM) having a name and an intensity of a compound included in an odor for each of the plurality of gas samples; and
generating the sample compound matrix and the electronic nose analysis result of each of the plurality of gas samples, included in the sample compound matrix, as the basic data.

19. The method of claim 18, wherein:
the sample compound matrix includes at least two compounds for each of the plurality of samples, and
the generating the basic data comprises:
selecting a main compound from among the compounds included in each of the plurality of gas samples, based on the electronic nose analysis result of each of the plurality of gas samples; and
generating the electronic nose analysis result and the main compound as the basic data.

20. The method of claim 19, wherein the generating the basic data comprises selecting a compound, selected repeatedly in at least two feature selection algorithms, as the main compound.
